(19) United States
(12) Patent Application Publication
(10) Pub. No.: US 2024/0225515 A9
(43) Pub. Date: Jul. 11, 2024

(54) STATE ESTIMATION APPARATUS, STATE ESTIMATION METHOD AND PROGRAM

(71) Applicant: Nippon Telegraph and Telephone Corporation, Tokyo (JP)

(72) Inventors: Kentaro TANAKA, Musashino-shi, Tokyo (JP); Shingo TSUKADA, Musashino-shi, Tokyo (JP); Masumi YAMAGUCHI, Musashino-shi, Tokyo (JP); Takayuki OGASAWARA, Musashino-shi, Tokyo (JP); Toichiro GOTO, Musashino-shi, Tokyo (JP)

(21) Appl. No.: 18/279,531

(22) PCT Filed: Mar. 5, 2021
(86) PCT No.: PCT/JP2021/008780

§ 371 Date: Aug. 30, 2023

(51) Int. Cl. A61B 5/361

(52) U.S. Cl. CPC A61B 5/361

(57) ABSTRACT

A state estimation device includes an information acquisition unit acquiring at least one of (i) electrode acquisition information indicating an electromagnetic time series that is a time series of an electromagnetic quantity that is an electromagnetic force, field, or energy applied to an electrode from which a signal indicating a state of a heart of an estimation target is acquirable or (ii) environment information that is information on a time series of an environmental quantity that is an amount related to one or both of a state of motion of the estimation target and an environment in which the estimation target is present; and an estimation unit using at least one of the electrode acquisition information or the environment information to determine whether an electrode event, which is an abnormality in acquisition of a signal indicating the state of the heart of the estimation target by the electrode, has occurred.

### TECHNICAL FIELD

The present invention relates to a state estimation device, a state estimation method, and a program.

### BACKGROUND ART

A technology for measuring a biosignal indicating a state of a heart by using a bioelectrode has been studied.

### CITATION LIST

### Non Patent Literature

Non Patent Literature 1: Kasai, Ogasawara, Nakashima, and Tsukada, “Development and practical use of functional material ‘hitoe’ that enables biological information measurement just by being worn” Communication Society Magazine No. 41, The Institute of Electronics, Information and Communication Engineers (June 2017) (Vol. 11 No. 1)

### SUMMARY OF INVENTION

### Technical Problem

However, in measurement using a bioelectrode performed non-invasively, friction or peeling occurs between the bioelectrode and the skin due to the body motion of the subject, which may cause deterioration in quality of the biosignal. The deterioration in quality appears in the biosignal as noise, and causes erroneous detection when event detection is performed using the biosignal. The event is occurrence of any one or both of an abnormal state of the heart such as ventricular fibrillation and an abnormality of measurement by the bioelectrode such as a phenomenon in which the bioelectrode is detached.

For example, in the electrocardiogram measurement, an abnormal waveform on a spike may be recorded due to a biological electrode contact abnormality during an interval of an electrocardiographic waveform which is originally observed with a constant rhythm. In such a case, there is a possibility that a cardiac abnormality event indicating an irregular heartbeat has occurred. However, when the quality has deteriorated, there may not be a result showing occurrence of a cardiac abnormality event but a result showing inappropriate contact between the bioelectrode and the skin, such as peeling or friction.

In view of the above circumstances, an object of the present invention is to provide a technology for improving the accuracy of estimation of the state of the heart.

### Solution to Problem

According to an aspect of the present invention, there is provided a state estimation device including: an information acquisition unit that acquires one or both of electrode acquisition information indicating an electromagnetic time series that is a time series of an electromagnetic quantity that is an electromagnetic force, field, or energy applied to an electrode from which a signal indicating a state of a heart of an estimation target is acquirable, and environment information that is information on a time series of an environmental quantity that is an amount related to one or both of a state of motion of the estimation target and an environment in which the estimation target is present; and an estimation unit that uses one or both of the electrode acquisition information and the environment information to determine whether or not an electrode event, which is an abnormality in acquisition of a signal indicating the state of the heart of the estimation target by the electrode, has occurred.

According to another aspect of the present invention, there is provided a state estimation method including: an information acquisition step of acquiring one or both of electrode acquisition information indicating an electromagnetic time series that is a time series of an electromagnetic quantity that is an electromagnetic force, field, or energy applied to an electrode from which a signal indicating a state of a heart of an estimation target is acquirable, and environment information that is information on a time series of an environmental quantity that is an amount related to one or both of a state of motion of the estimation target and an environment in which the estimation target is present; and an estimation step of using one or both of the electrode acquisition information and the environment information to determine whether or not an electrode event, which is an abnormality in acquisition of a signal indicating the state of the heart of the estimation target by the electrode, has occurred.

According to still another aspect of the present invention, there is a program for causing a computer to function as the above-described state estimation device.

### Advantageous Effects of Invention

According to the present invention, the accuracy of the estimation of the state of the heart can be improved.

### BRIEF DESCRIPTION OF DRAWINGS

FIG. 1 is an explanatory diagram for explaining an outline of an abnormal state estimation system 100 according to an embodiment.

FIG. 2 is an explanatory diagram for explaining biosignal abnormality amplitude determination processing according to the embodiment.

FIG. 3 is an explanatory diagram for explaining biosignal abnormal standard deviation determination processing according to the embodiment.

FIG. 4 is a diagram for explaining an example of a lapse of time of estimation reliability according to the embodiment.

FIG. 5 is a diagram illustrating an example of a hardware configuration of a monitoring device 4 according to the embodiment.

FIG. 6 is a diagram illustrating an example of a functional configuration of a control unit 41 according to the embodiment.

FIG. 7 is a diagram illustrating an example of a hardware configuration of a control device 5 according to the embodiment.

FIG. 8 is a diagram illustrating an example of a functional configuration of a control unit 51 according to the embodiment.

FIG. 9 is a flowchart illustrating an example of a flow of processing executed by the abnormal state estimation system 100 according to the embodiment.

FIG. 10 is a diagram illustrating an example of a cardiac state quantity time series obtained from the heart which is in a normal state according to the embodiment.

FIG. 11 is a diagram illustrating an example of a cardiac state quantity time series obtained from the heart which is in an abnormal state according to the embodiment.

FIG. 12 is a diagram illustrating an upper threshold value, a lower threshold value, a threshold region, and out-of-range data according to the embodiment.

FIG. 13 is an explanatory diagram for explaining an effect obtained by fourth-type cardiac state estimation processing according to a modification example.

### DESCRIPTION OF EMBODIMENTS

### Embodiment

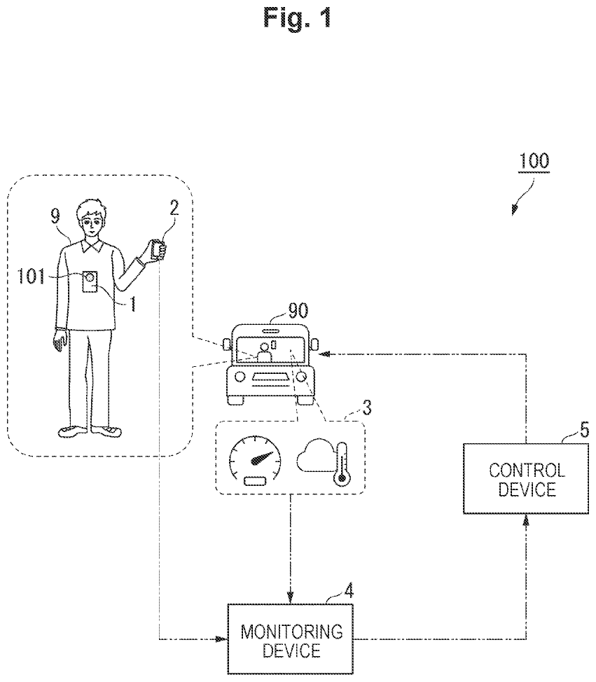

FIG. 1 is an explanatory diagram for explaining an outline of an abnormal state estimation system 100 according to an embodiment. The abnormal state estimation system 100 determines whether or not an electrode event has occurred in an electrode 101 that acquires a biosignal indicating a state of the heart of an estimation target 9. The biosignal indicating the state of the heart is, for example, a time series of cardiac potentials. The biosignal indicating the state of the heart is, for example, a time series of heart rate. The electrode event is an abnormality in acquisition of a signal indicating the state of the heart of the estimation target 9 by the electrode 101, such as a phenomenon in which the electrode 101 deviates from the estimation target 9.

The abnormal state estimation system 100 determines whether or not a cardiac abnormality event has occurred. The cardiac abnormality event is an abnormality in the state of the heart of the estimation target 9. The abnormality of the state of the heart is, for example, ventricular fibrillation.

The abnormal state estimation system 100 estimates the probability of the estimation result of the cardiac abnormality event.

The estimation target 9 may be any living body as long as the living body has a heart, and is, for example, a human. The estimation target 9 may be an animal other than a human. The estimation target 9 includes the biosignal acquisition device 1.

The biosignal acquisition device 1 includes the electrode 101. The biosignal acquisition device 1 acquires information via the electrode 101. The electrode 101 is an electrode that can be attached to the estimation target 9. The electrode 101 can acquire information indicating the state of the heart of the estimation target 9. The information acquired by the electrode 101 when the electrode 101 is attached to the estimation target 9 indicates the state of the heart of the estimation target 9. Specifically, the information acquired by the electrode 101 is a signal such as a biosignal or noise applied to the electrode 101.

Hereinafter, the information acquired by the biosignal acquisition device 1 via the electrode 101 is referred to as electrode acquisition information. The electrode acquisition information is information on a time series (hereinafter referred to as “electromagnetic time series”) of the electromagnetic force, field, or energy (hereinafter referred to as “electromagnetic quantity”) applied to the electrode 101. The electromagnetic quantity is, for example, an electromagnetic field. The electromagnetic quantity may be, for example, a voltage. The electromagnetic quantity may be, for example, a current.

Hereinafter, the time series of the electromagnetic quantity applied to the electrode 101 when the electrode 101 is attached to the estimation target 9 is referred to as a cardiac state time series. Since the cardiac state time series is a time series of the electromagnetic quantity applied to the electrode 101 when the electrode 101 is attached to the estimation target 9, the cardiac state time series is a time series of a quantity (hereinafter referred to as a “cardiac state quantity”) indicating the state of the heart of the estimation target 9. Hereinafter, the information indicating the cardiac state time series is referred to as cardiac state information.

Since the biosignal indicating the state of the heart of the estimation target 9 is a signal acquired by the electrode 101 when the electrode 101 is attached to the estimation target 9, the biosignal indicating the state of the heart of the estimation target 9 is information indicating a cardiac state time series (that is, cardiac state information).

Thus, the cardiac state information is a type of electrode acquisition information, the cardiac state quantity is a type of electromagnetic quantity, and the cardiac state time series is a type of electromagnetic time series.

The biosignal acquisition device 1 is, for example, a device capable of acquiring cardiac state information, and is a wearable device worn by the estimation target 9. The biosignal acquisition device 1 is a device including a cardiac potential sensor that detects a cardiac potential from the estimation target 9 via, for example, a conductive electrode. The biosignal acquisition device 1 repeatedly acquires the cardiac state quantity of the estimation target 9 at predetermined time intervals.

The estimation target 9 in the abnormal state estimation system 100 may be in a moving object such as an automobile 90. Hereinafter, for simplicity of explanation, the abnormal state estimation system 100 will be described by taking, as an example, a case of estimating an abnormality of the heart of the estimation target 9 while riding in the automobile 90.

The abnormal state estimation system 100 includes a biosignal acquisition device 1, a relay terminal 2, an environment sensor 3, a monitoring device 4, and a control device 5. The biosignal acquisition device 1 outputs the acquired electrode acquisition information to the relay terminal 2.

The relay terminal 2 is a device that transmits the electrode acquisition information acquired by the biosignal acquisition device 1 to the monitoring device 4. The relay terminal 2 is, for example, a device including an antenna that transmits electrode acquisition information. The relay terminal 2 may be, for example, a mobile terminal such as a smartphone or a tablet that acquires and transmits electrode acquisition information from the biosignal acquisition device 1.

The relay terminal 2 converts, for example, the electrode acquisition information from an analog signal to a digital signal. Note that the electrode acquisition information does not necessarily need to be transmitted from the relay terminal 2 in the form of a digital signal, and may be transmitted in the form of an analog signal. Note that the conversion of the electrode acquisition information from the analog signal to the digital signal is not necessarily executed by the relay terminal 2, and may be executed by the biosignal acquisition device 1. The conversion of the electrode acquisition information from the analog signal to the digital signal may be executed by the monitoring device 4. For simplicity of explanation, the abnormal state estimation system 100 will be described by taking, as an example, a case where the electrode acquisition information is transmitted from the relay terminal 2 in the form of a digital signal.

The environment sensor 3 is a sensor that acquires information on a time series (hereinafter referred to as “environment information”) of a quantity (hereinafter referred to as “environmental quantity”) regarding one or both of the motion state of the estimation target 9 and the environment in which the estimation target 9 is located. The environment sensor 3 is, for example, a speedometer that measures the moving speed of the estimation target 9. In such a case, the environment information indicates a time series of the moving speed of the estimation target 9. The environment sensor 3 may be, for example, a temperature sensor that measures the temperature of the space in which the estimation target 9 is located. In such a case, the environment information indicates a time series of the temperature of the space in which the estimation target 9 is located.

The environment sensor 3 may be, for example, a sensor that measures the state of body motion of the estimation target 9. The state of body motion of the estimation target 9 is, for example, acceleration of movement of the estimation target 9. In such a case, the environment information indicates a time series of acceleration of the movement of the estimation target 9. Note that the environment sensor 3 does not necessarily indicate only one type of information, and may indicate a plurality of types of information. For example, the environment sensor 3 may indicate a time series of the moving speed of the estimation target 9 and the temperature of the space in which the estimation target 9 is located.

The environment sensor 3 is, for example, a sensor mounted on the automobile 90, and may be a sensor that acquires information (hereinafter referred to as “in-vehicle information”) indicating the state of the automobile 90 such as an acceleration sensor, a temperature sensor, or a speedometer mounted on the automobile 90. The in-vehicle information is an example of the environment information.

The environment sensor 3 may be, for example, an acceleration sensor. Note that the environment sensor 3 is not necessarily mounted as a device different from the biosignal acquisition device 1, and may be provided in the biosignal acquisition device 1. The environment sensor 3 may be mounted as a device worn by the estimation target 9 or may be provided in the automobile 90 in which the estimation target 9 is riding.

The environment sensor 3 transmits the acquired environment information to the monitoring device 4.

The monitoring device 4 acquires at least electrode acquisition information. The monitoring device 4 executes cardiac abnormality event occurrence determination processing. The cardiac abnormality event occurrence determination processing is processing of determining whether or not a cardiac abnormality event has occurred based on at least the electrode acquisition information. The cardiac abnormality event occurrence determination processing may be any processing as long as it is possible to determine whether or not a cardiac abnormality event has occurred based on at least the electrode acquisition information. The cardiac abnormality event occurrence determination processing is processing of executing cardiac state estimation processing to be described later, for example, and determining that a cardiac abnormality event has occurred when the execution result indicates that the state of the heart of the estimation target 9 is abnormal.

The monitoring device 4 executes electrode event occurrence determination processing. The electrode event occurrence determination processing is processing of determining whether or not an electrode event has occurred based on one or both of the electrode acquisition information and the environment information. Hereinafter, the electrode event occurrence determination processing will be described by taking, as an example, a case where occurrence of an electrode event is determined using only the electrode acquisition information for simplicity of description. The electrode event occurrence determination processing using the environment information will be described in a modification example.

The monitoring device 4 executes estimation reliability estimation processing. The estimation reliability estimation processing includes estimating the probability (hereinafter referred to as “estimation reliability”) of the estimation result of the cardiac abnormality event occurrence determination processing based on at least the determination result of the electrode event occurrence determination processing.

Based on the determination result and the estimation result of the monitoring device 4, the control device 5 determines whether or not the determination result and the estimation result of the monitoring device 4 satisfy a notification criterion that is a predetermined criterion. Specifically, the notification criterion is a predetermined criterion for determining whether to notify a predetermined notification destination of the determination result and the estimation result of the monitoring device 4 regarding the state of the heart of the estimation target 9. When the determination result and the estimation result satisfy the warning notification criterion, the control device 5 notifies a predetermined notification destination that the heart of the estimation target 9 is abnormal. Hereinafter, processing of determining whether or not the determination result and the estimation result of the monitoring device 4 satisfy the notification criterion is referred to as notification determination processing.

### <Description of Example of Electrode Event Occurrence Determination Processing>

The electrode event occurrence determination processing includes biosignal abnormality amplitude determination processing, biosignal abnormality standard deviation determination processing, and electrode state determination processing.

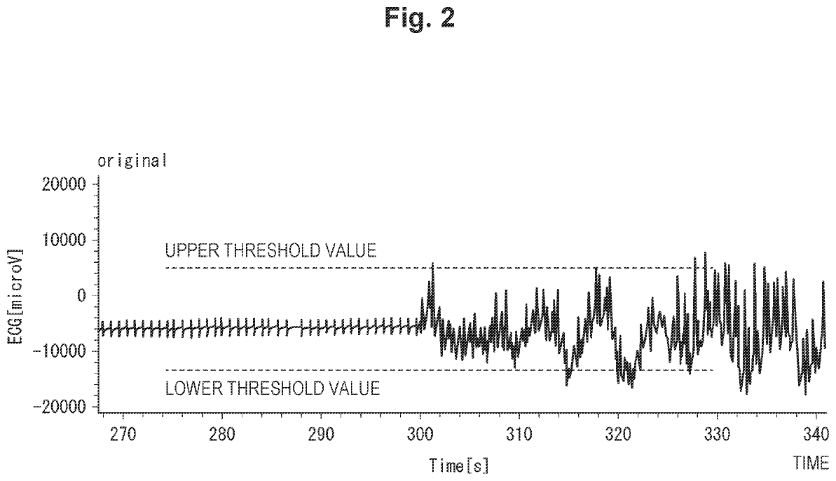

FIG. 2 is an explanatory diagram for explaining biosignal abnormality amplitude determination processing according to the embodiment. The biosignal abnormality amplitude determination processing is processing of determining a timing at which the electromagnetic quantity indicated by the electromagnetic time series exceeds a predetermined upper limit value and a timing at which the electromagnetic quantity falls below a predetermined lower limit value as timing of a candidate for an electrode event (hereinafter referred to as “electrode event candidates”). The graph of FIG. 2 is an example of electromagnetic time series. In FIG. 2, the horizontal axis represents time, and the vertical axis represents electromagnetic quantity.

The upper limit value and the lower limit value in the biosignal abnormality amplitude determination processing may be predetermined values or values determined based on the electromagnetic time series.

The upper limit value and the lower limit value in the biosignal abnormality amplitude determination processing are, for example, absolute values of the electromagnetic time series. The upper limit value and the lower limit value in the biosignal abnormality amplitude determination processing may be, for example, relative values from a reference value calculated from the average behavior of the electromagnetic time series in a predetermined period immediately before the biosignal abnormality amplitude determination processing is executed. The predetermined period is, for example, a period of several seconds. The predetermined period is a period of several tens of seconds.

The average behavior is a state of the electromagnetic time series included in a predetermined period. The average behavior is expressed by an output of a predetermined function having an electromagnetic time series included in a predetermined period as an input and an electromagnetic output amount as an output. The electromagnetic output amount is an amount indicating a state of electromagnetic time series for a predetermined period. The amount indicating the state of the electromagnetic time series in the predetermined period is, for example, a statistic such as an average value, a standard deviation, or an intermediate value.

The reference value may be, for example, an average value of electromagnetic time series for a predetermined period. However, the reference value is not limited to the average value as long as the reference value is an amount based on the reference value for the predetermined period.

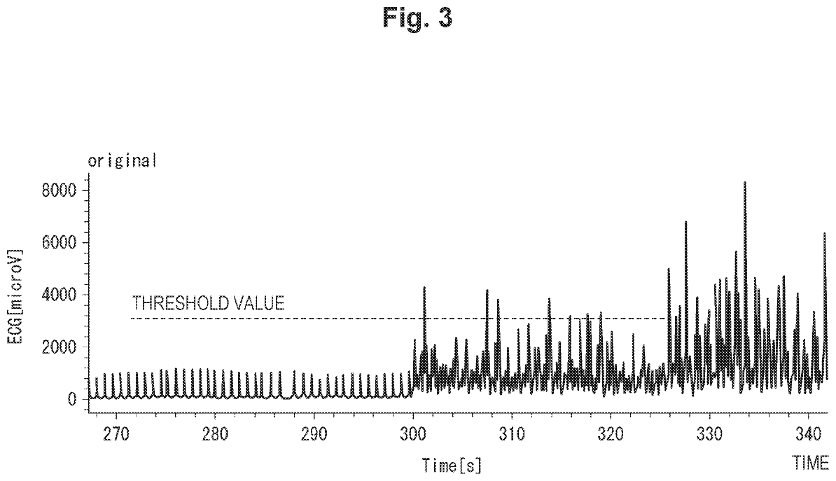

FIG. 3 is an explanatory diagram for explaining biosignal abnormal standard deviation determination processing according to the embodiment. The graph of FIG. 3 is an example of the standard deviation (hereinafter referred to as “electromagnetic deviation”) of the distribution of the electromagnetic quantity indicated by the electromagnetic time series. In FIG. 3, the horizontal axis represents time, and the vertical axis represents the standard deviation of the distribution of the electromagnetic quantity indicated by the electromagnetic time series. The biosignal abnormality standard deviation determination processing is processing of determining the timing at which the electromagnetic deviation exceeds a predetermined threshold value as the timing of the electrode event candidate.

The predetermined threshold value in the biosignal abnormal standard deviation determination processing may be a predetermined value or a value determined based on the electromagnetic time series.

The predetermined threshold value in the biosignal abnormal standard deviation determination processing is, for example, an absolute value of the electromagnetic deviation. The predetermined threshold value in the biosignal abnormal standard deviation determination processing may be, for example, a relative value from a reference value calculated from the average behavior of the electromagnetic deviation in a predetermined period immediately before the biosignal abnormality amplitude determination processing is executed. The predetermined period is, for example, a period of several seconds. The predetermined period is a period of several tens of seconds.

The reference value may be, for example, an average value of electromagnetic time series for a predetermined period. However, the reference value is not limited to the average value as long as the reference value is an amount based on the reference value for the predetermined period.

The electrode state determination processing is processing of determining that the timing at which one or both of the result of the biosignal abnormality amplitude determination processing and the result of the biosignal abnormal standard deviation determination processing are determined to be the timing of the electrode event candidate is the timing of occurrence of the electrode event.

### <Description of Example of Estimation Reliability Estimation Processing>

An example of specific processing of the estimation reliability estimation processing will be described. In the estimation reliability estimation processing, when it is determined that a cardiac abnormality event has occurred in the cardiac abnormality event occurrence determination processing, processing of lowering the estimation reliability to a predetermined lower limit value is executed. In the estimation reliability estimation processing, processing of not changing the estimation reliability is executed for a certain period of time after the processing of lowering the estimation reliability to a predetermined lower limit value is executed.

In the estimation reliability estimation processing, when it is determined that a cardiac abnormality event has occurred by the cardiac abnormality event occurrence determination processing after a lapse of a certain period of time, processing of increasing the reliability by a predetermined amount is executed. In the estimation reliability estimation processing, when it is determined that no cardiac abnormality event has occurred by the cardiac abnormality event occurrence determination processing after a lapse of a certain period of time, processing of decreasing the reliability by a predetermined amount is executed. However, it is desirable that the reliability increased in one determination be a predetermined amount.

In addition, in the estimation reliability estimation processing, in a case where it is determined by the cardiac abnormality event occurrence determination processing that no cardiac abnormality event has occurred after a certain period of time has elapsed, the reliability is increased every time the certain period of time has elapsed.

In the estimation reliability estimation processing, when it is determined by the electrode event occurrence determination processing that an electrode event has occurred, the processing of stopping the processing of determining a cardiac abnormality event and lowering the estimation reliability is executed for a certain period of time.

In the estimation reliability estimation processing, in a case where it is determined that no electrode event has occurred by the electrode event occurrence determination processing, processing of not changing the reliability is executed.

In the estimation reliability estimation processing, the reliability is gradually increased in a case where no electrode event occurs.

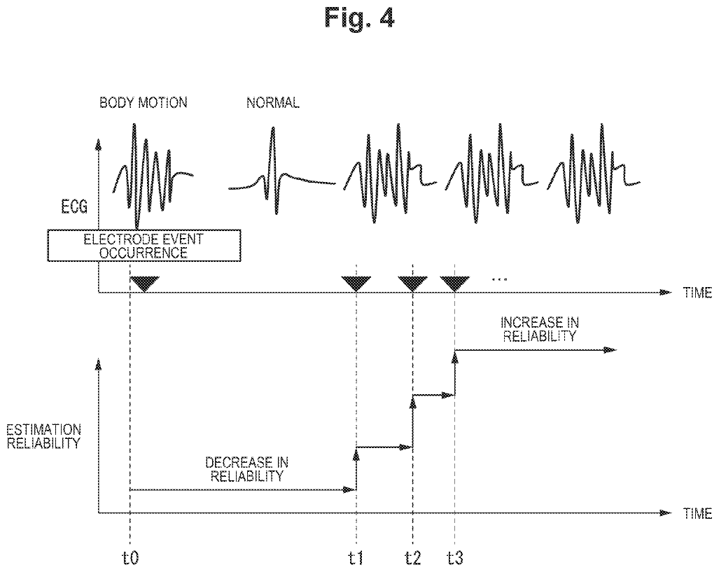

FIG. 4 is a diagram for explaining an example of a lapse of time of estimation reliability according to the embodiment. FIG. 4 illustrates that it is determined that an electrode event has occurred at time t0 by the electrode event occurrence determination processing. FIG. 4 illustrates that it is determined that a cardiac abnormality event has occurred at time t1 by the cardiac abnormality event occurrence determination processing. FIG. 4 illustrates that it is determined that a cardiac abnormality event has occurred at time t2 by the cardiac abnormality event occurrence determination processing. FIG. 4 illustrates that it is determined that a cardiac abnormality event has occurred at time t3 by the cardiac abnormality event occurrence determination processing. FIG. 4 illustrates that the estimation reliability increases as time elapses after time t1.

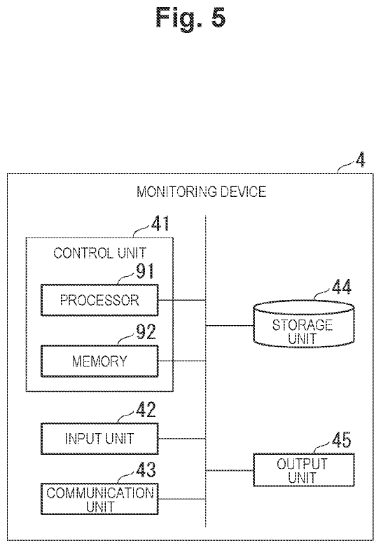

FIG. 5 is a diagram illustrating an example of a hardware configuration of the monitoring device 4 according to the embodiment. The monitoring device 4 includes a control unit 41 including a processor 91 such as a central processing unit (CPU) and a memory 92, which are connected by a bus, and executes a program. The monitoring device 4 functions as a device including the control unit 41, an input unit 42, a communication unit 43, a storage unit 44, and an output unit 45 by executing the program.

More specifically, the processor 91 reads the program stored in the storage unit 44, and stores the read program in the memory 92. The processor 91 executes the program stored in the memory 92, whereby the monitoring device 4 functions as a device including the control unit 41, the input unit 42, the communication unit 43, the storage unit 44, and the output unit 45.

The control unit 41 controls operation of various functional units included in the monitoring device 4. The control unit 41 executes, for example, electrode event occurrence determination processing. The control unit 41 executes, for example, cardiac abnormality event occurrence determination processing. The control unit 41 executes, for example, estimation reliability estimation processing.

The control unit 41 controls, for example, the operation of the output unit 45. The control unit 41 records, in the storage unit 44, various types of information generated by execution of, for example, electrode event occurrence determination processing, cardiac abnormality event occurrence determination processing, or estimation reliability estimation processing. The control unit 41 records, for example, the electrode acquisition information input to the input unit 42 or the communication unit 43 in the storage unit 44.

The input unit 42 includes an input device such as a mouse, a keyboard, or a touch panel. The input unit 42 may be configured as an interface that connects these input devices to the monitoring device 4. The input unit 42 receives inputs of various types of information to the monitoring device 4. For example, electrode acquisition information is input to the input unit 42. For example, environment information may be input to the input unit 42.

The communication unit 43 includes a communication interface for connecting the monitoring device 4 to an external device. The communication unit 43 communicates with an external device in a wired or wireless manner. The external device is, for example, a device which is a transmission source of the electrode acquisition information. The transmission source of the electrode acquisition information is, for example, the relay terminal 2. The external device is, for example, the control device 5. The communication unit 43 may communicate with the environment sensor 3. In a case where the communication unit 43 communicates with the environment sensor 3, the communication unit 43 may acquire the environment information acquired by the environment sensor 3 by communication with the environment sensor 3.

The storage unit 44 is configured using a computer-readable storage medium device such as a magnetic hard disk device or a semiconductor storage device. The storage unit 44 stores various types of information related to the monitoring device 4. The storage unit 44 stores, for example, information input via the input unit 42 or the communication unit 43. The storage unit 44 stores, for example, various types of information generated by execution of electrode event occurrence determination processing, cardiac abnormality event occurrence determination processing, or estimation reliability estimation processing. The storage unit 44 stores, for example, the estimation reliability. The storage unit 44 stores, for example, a history of estimation reliability.

Note that the electrode acquisition information and the environment information do not necessarily need to be input only to the input unit 42, and does not need to be input only to the communication unit 43. The electrode acquisition information and the environment information may be input from either the input unit 42 or the communication unit 43.

The output unit 45 outputs various types of information. The output unit 45 includes a display device such as a cathode ray tube (CRT) display, a liquid crystal display, or an organic electro-luminescence (EL) display, for example. The output unit 45 may be configured as an interface that connects these display devices to the monitoring device 4. The output unit 45 outputs, for example, information input to the input unit 42. The output unit 45 may display, for example, the execution results of the electrode event occurrence determination processing and the cardiac abnormality event occurrence determination processing. The output unit 45 may output, for example, the estimation reliability. The output unit 45 may output, for example, a history of the estimation reliability.

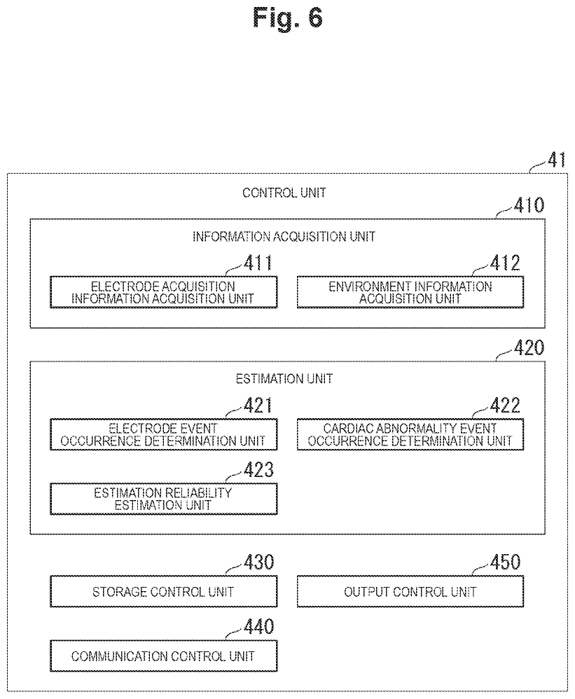

FIG. 6 is a diagram illustrating an example of a functional configuration of the control unit 41 according to the embodiment. The control unit 41 includes an information acquisition unit 410, an estimation unit 420, a storage control unit 430, a communication control unit 440, and an output control unit 450.

The information acquisition unit 410 includes an electrode acquisition information acquisition unit 411 and an environment information acquisition unit 412. The electrode acquisition information acquisition unit 411 repeatedly acquires the electrode acquisition information at a predetermined cycle via the input unit 42 or the communication unit 43. That is, the electrode acquisition information acquisition unit 411 acquires the electrode acquisition information.

The environment information acquisition unit 412 repeatedly acquires environment information at a predetermined cycle via the input unit 42 or the communication unit 43. That is, the environment information acquisition unit 412 acquires the environment information. Note that the information acquisition unit 410 does not necessarily need to acquire both the electrode acquisition information and the environment information, and may acquire either one.

The estimation unit 420 includes an electrode event occurrence determination unit 421, a cardiac abnormality event occurrence determination unit 422, and an estimation reliability estimation unit 423. The electrode event occurrence determination unit 421 executes the electrode event occurrence determination processing. The cardiac abnormality event occurrence determination unit 422 executes the cardiac abnormality event occurrence determination processing. The estimation reliability estimation unit 423 executes the estimation reliability estimation processing.

The storage control unit 430 records various types of information in the storage unit 44. The communication control unit 440 controls an operation of the communication unit 43. The communication control unit 440 controls the operation of the communication unit 43 to cause the communication unit 43 to transmit, for example, the result obtained by the estimation unit 420 to the control device 5. The output control unit 450 controls the operation of the output unit 45. For example, the output control unit 450 controls the operation of the output unit 45 to cause the output unit 45 to output the result obtained by the estimation unit 420. The result that the output control unit 450 causes the output unit 45 to output is, for example, the estimation reliability.

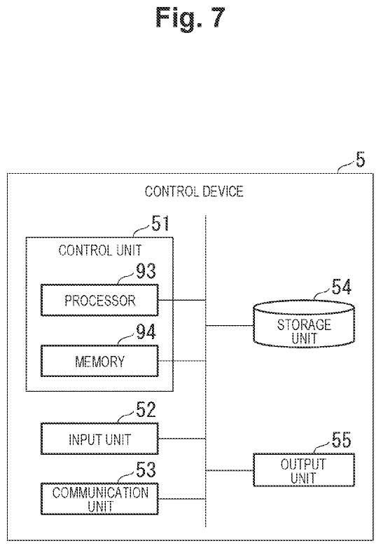

FIG. 7 is a diagram illustrating an example of a hardware configuration of the control device 5 according to the embodiment. The control device 5 includes a control unit 51 including a processor 93 such as a CPU and a memory 94 connected by a bus, and executes a program. The control device 5 functions as a device including the control unit 51, an input unit 52, a communication unit 53, a storage unit 54, and an output unit 55 by executing the program.

More specifically, the processor 93 reads the program stored in the storage unit 44, and stores the read program in the memory 94. The processor 93 executes the program stored in the memory 94, whereby the control device 5 functions as a device including the control unit 51, the input unit 52, the communication unit 53, the storage unit 54, and the output unit 55.

The control unit 51 controls operations of various functional units included in the control device 5. The control unit 51 executes, for example, notification determination processing. The control unit 51 controls, for example, the operation of the communication unit 53. The control unit 51 controls, for example, the operation of the communication unit 53 to transmit the notification to the notification destination. The control unit 51 controls, for example, the operation of the output unit 55. The control unit 51 records, for example, various types of information generated by execution of the notification determination processing in the storage unit 54. The control unit 51 stores, for example, information input to the input unit 52 or the communication unit 53 in the storage unit 54. The information input to the input unit 52 or the communication unit 53 is, for example, a result obtained by the estimation unit 420.

The input unit 52 includes an input device such as a mouse, a keyboard, or a touch panel. The input unit 52 may be configured as an interface that connects these input devices to the control device 5. The input unit 52 receives inputs of various types of information to the control device 5. For example, a result obtained by the estimation unit 420 is input to the input unit 52.

The communication unit 53 includes a communication interface for connecting the control device 5 to an external device. The communication unit 53 communicates with an external device in a wired or wireless manner. The external device is, for example, the monitoring device 4. The external device is, for example, a predetermined notification destination.

The storage unit 54 is configured using a computer-readable storage medium device such as a magnetic hard disk device or a semiconductor storage device. The storage unit 54 stores various types of information related to the control device 5. The storage unit 54 stores, for example, information input via the input unit 52 or the communication unit 53. The storage unit 54 stores, for example, various types of information generated by execution of the notification determination processing.

Note that the result obtained by the estimation unit 420 (that is, the result obtained by the monitoring device 4) does not necessarily need to be input only to the input unit 52, and does not need to be input only to the communication unit 53. The result obtained by the estimation unit 420 may be input from either the input unit 52 or the communication unit 53.

The output unit 55 outputs various types of information. The output unit 55 includes, for example, a display device such as a CRT display, a liquid crystal display, or an organic EL display. The output unit 55 may be configured as an interface that connects these display devices to the control device 5. The output unit 55 outputs, for example, information input to the input unit 52. The output unit 55 may display, for example, the estimation result input to the input unit 52 or the communication unit 53. The output unit 55 may display, for example, an execution result of the notification determination processing.

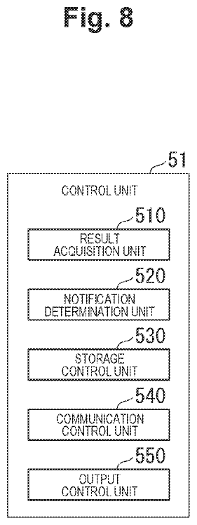

FIG. 8 is a diagram illustrating an example of a functional configuration of the control unit 51 according to the embodiment. The control unit 51 includes a result acquisition unit 510, a notification determination unit 520, a storage control unit 530, a communication control unit 540, and an output control unit 550.

The result acquisition unit 510 repeatedly acquires the result obtained by the estimation unit 420 input to the input unit 52 or the communication unit 53 at a predetermined cycle.

The notification determination unit 520 executes the notification determination processing on the result acquired by the result acquisition unit 510. That is, the notification determination unit 520 determines whether or not the result acquired by the result acquisition unit 510 satisfies the notification criterion. More specifically, the target result of the notification determination processing includes at least a result of determination as to whether or not a cardiac abnormality event has occurred.

The storage control unit 530 records various types of information in the storage unit 54. The communication control unit 540 controls the operation of the communication unit 53.

The communication control unit 540 controls the operation of the communication unit 53 to cause the communication unit 53 to execute, for example, notification to a notification destination. The communication control unit 540 may cause the communication unit 53 to transmit a control signal for controlling the operation of the automobile 90, such as a signal for instructing the automobile 90 to decelerate or a signal for instructing the automobile 90 to stop.

The output control unit 550 controls the operation of the output unit 55. For example, the output control unit 550 controls the operation of the output unit 55 to cause the output unit 55 to output the determination result of the notification determination unit 520.

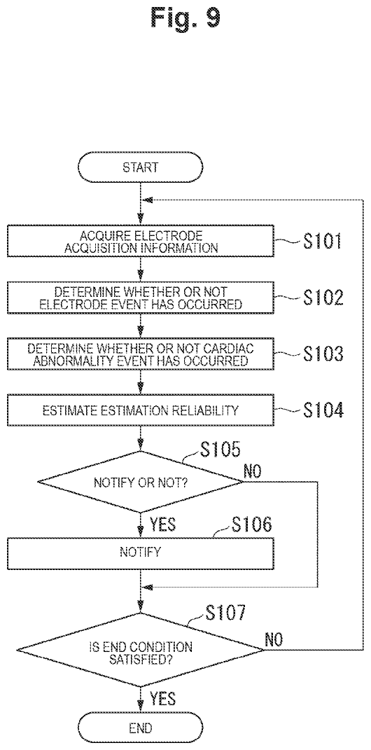

FIG. 9 is a flowchart illustrating an example of a flow of processing executed by the abnormal state estimation system 100 according to the embodiment. The abnormal state estimation system 100 repeatedly executes the processing illustrated in the flowchart illustrated in FIG. 9 until a predetermined end condition is satisfied. The predetermined end condition is, for example, a condition that power supply to the biosignal acquisition device 1 is interrupted. For example, the control unit 41 determines whether or not the end condition is satisfied. For example, the estimation unit 420 determines whether or not the end condition is satisfied. For example, the notification determination unit 520 may determine whether or not the end condition is satisfied.

The information acquisition unit 410 acquires the electrode acquisition information acquired via the electrode 101 (step S101). Next, the electrode event occurrence determination unit 421 determines whether or not an electrode event has occurred (step S102). Next, the cardiac abnormality event occurrence determination unit 422 determines whether or not a cardiac abnormality event has occurred (step S103). Next, the estimation reliability estimation unit 423 estimates the estimation reliability (step S104). Next, the notification determination unit 520 determines whether to notify the notification destination based on the results obtained in steps 3102 to 3104 (step S105).

When it is determined to notify the notification destination (step 3105: YES), the communication control unit 540 controls the operation of the communication unit 53 to notify the notification destination (step S106). After step S106, it is determined whether or not the end condition is satisfied (step S107). When the end condition is satisfied (step S107: YES), the processing ends. When the end condition is not satisfied (step S107: NO), the processing returns to step S101.

In a case where it is determined not to notify the notification destination (step S105: NO), the processing of step S107 is executed.

The abnormal state estimation system 100 according to the embodiment configured as described above determines whether or not the electrode 101 is detached. Therefore, when the abnormal state estimation system 100 determines that the electrode 101 is detached, the user using the abnormal state estimation system 100 is likely to reconfirm whether or not the estimation result of the state of the heart of the estimation target 9 is correct. That is, since the abnormal state estimation system 100 determines whether or not the electrode 101 is detached, it is possible to increase the frequency of causing the user to reconfirm the correctness of the estimation result. Therefore, the abnormal state estimation system 100 can improve the accuracy of the estimation of the state of the heart.

In addition, the abnormal state estimation system 100 according to the embodiment configured as described above estimates the probability of the estimation result. Therefore, the abnormal state estimation system 100 can improve the accuracy of the estimation of the state of the heart.

Here, an example of the cardiac state estimation processing will be described.

### <Description of Cardiac State Estimation Processing>

The cardiac state estimation processing is, for example, the following first-type cardiac state estimation processing. The first-type cardiac state estimation processing includes statistic calculation processing and abnormality estimation processing. The statistic calculation processing is repeatedly executed at a predetermined cycle. Hereinafter, a length of one cycle of a cycle in which the statistic calculation processing is executed is referred to as a unit processing period. The length of the unit processing period is, for example, 2 seconds.

The statistic calculation processing is processing of calculating statistic (hereinafter referred to as “electromagnetic statistic”) related to the electromagnetic time series indicated by the electrode acquisition information. The electromagnetic statistic is, for example, a time average of the electromagnetic quantity. The statistic of the electromagnetic time series is, for example, a deviation of the distribution of the electromagnetic quantity. The deviation may be any amount as long as the amount indicates a difference from the average value. Therefore, the deviation may be, for example, variance. The deviation may be, for example, a standard deviation.

In the statistic calculation processing, a statistic related to the electromagnetic time series is acquired using a sample satisfying a predetermined condition (hereinafter referred to as “sample conditions”). The sample condition is, for example, a condition that all the samples included in the electrode acquisition information acquired by the monitoring device 4 during the unit processing period immediately before the statistic calculation processing is executed. Therefore, for example, when the unit processing period is two seconds, the number of samples used for the statistic calculation processing is all the samples included in the electrode acquisition information acquired by the monitoring device 4 in the last two seconds.

The abnormality estimation processing is processing of estimating whether or not the state of the heart of the estimation target 9 is in an abnormal state. The abnormality of the estimation target by the abnormality estimation processing is, for example, ventricular fibrillation. The abnormality estimation processing includes non-responsive section sample determination processing, out-of-range data determination processing, and ventricular abnormality determination processing.

In order to facilitate understanding of the non-responsive section sample determination processing, the out-of-range data determination processing, and the ventricular abnormality determination processing, electromagnetic time series obtained from the heart which is in a normal state and electromagnetic time series obtained from the heart which is in an abnormal state will be described.

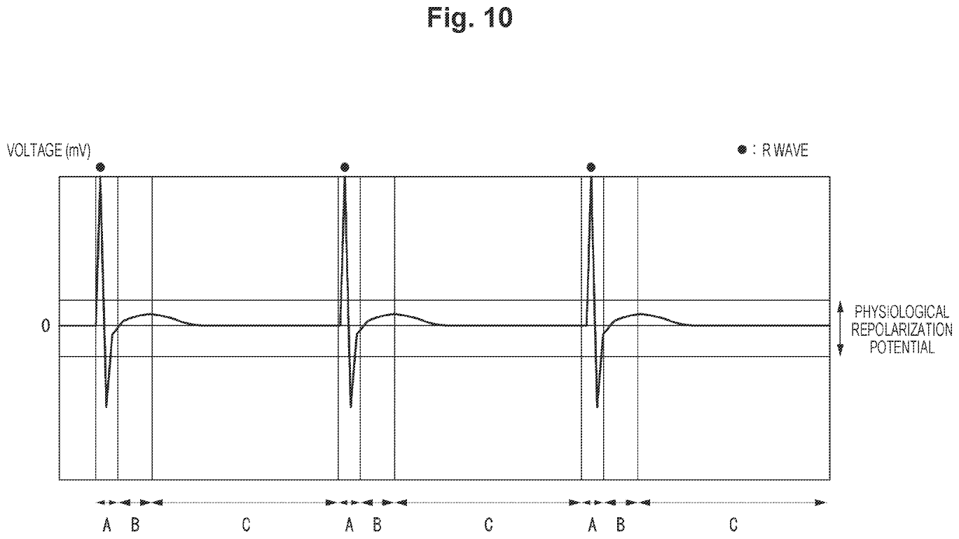

FIG. 10 is a diagram illustrating an example of the heart state quantity time series obtained from the heart which is in a normal state according to the embodiment. More specifically, FIG. 10 is a diagram illustrating an example of the time series of the cardiac potentials obtained from the heart which is in a normal state. In FIG. 10, the vertical axis represents the cardiac potential, and the horizontal axis represents time.

When the heart beats normally, an electrocardiographic waveform including an R wave is observed. A black circle in FIG. 10 indicates an R wave. A, B, and C in FIG. 10 each indicate the type of an activity period related to polarization when the heart beats. Hereinafter, the period of the type A is referred to as an A period. Hereinafter, the period of the type B is referred to as a B period. Hereinafter, the period of the type C is referred to as a C period.

The A period is a polarization section of the myocardium. In the A period, the R waveform is mainly observed. The B period is an absolute non-responsive interval. The B period is a period immediately after the myocardial polarization. In the B period, in principle of the myocardium, when the state of the heart is normal, there is no generation of the cardiac potential corresponding to the waveform. The C period is a relative non-responsive interval. In the C period, when the state of the heart is normal, there is no waveform due to a pulsatile trend of a constant rhythm. Note that, in a case where the B period and the C period are not distinguished from each other, the B period and the C period are generally referred to as a non-responsive section.

As described above, in the case of the time series of the cardiac potentials obtained from the normal heart, the A period, the B period, and the C period can be determined. In addition, in the case of the time series of the cardiac potential obtained from the normal heart, the voltage change from 0 millivolt is smaller in the non-responsive section of the cardiac potential (that is, the B period and the C period) than in the A period in which the R wave is generated. A range of a voltage change in the A period of a time series of cardiac potentials obtained from a normal heart is generally referred to as a range of a physiologically normal repolarization potential change.

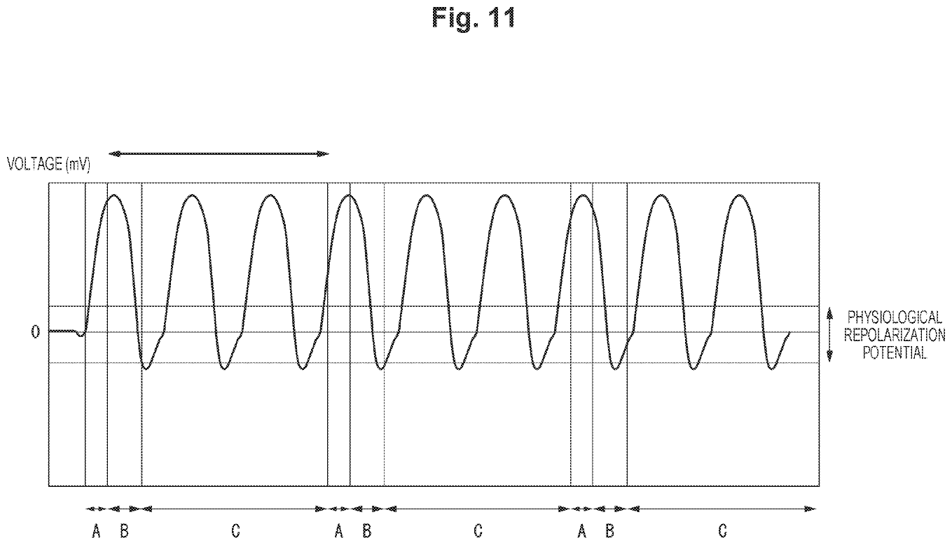

FIG. 11 is a diagram illustrating an example of the heart state quantity time series obtained from the heart which is in an abnormal state according to the embodiment. Specifically, FIG. 11 is a diagram illustrating an example of the time series of the cardiac potential obtained from the heart which is in an abnormal state. More specifically, FIG. 11 is a diagram illustrating an example of the time series of the cardiac potentials obtained from the heart which is in a state of ventricular fibrillation. In FIG. 11, the vertical axis represents the cardiac potential, and the horizontal axis represents time.

A, B, and C in FIG. 11 respectively indicate the A period, the B period, and the C period. FIG. 3 illustrates that, in the cardiac potential at the time of ventricular fibrillation, the behavior of the cardiac potential out of the range of the physiologically normal repolarization potential change occurs even in the period corresponding to the non-responsive section (that is, the B period and the C period) in the normal cardiac potential.

The abnormal state estimation system 100 estimates whether the state of the heart of the estimation target 9 is normal or abnormal based on the difference in behavior of the cardiac potential existing between the normal heart and the abnormal heart. The out-of-range data determination processing executed in the abnormal state estimation system 100 is a processing executed to quantify the degree of occurrence of out-of-range data in a section corresponding to a non-responsive section of a normal cardiac potential using a statistic.

Each of the non-responsive section sample determination processing, the out-of-range data determination processing, and the ventricular abnormality determination processing will be described.

The non-responsive section sample determination processing is processing of determining which sample belongs to the non-responsive section among the samples of the cardiac state time series. The non-responsive section sample determination processing is, for example, processing of determining a sample satisfying a predetermined condition as a sample belonging to the non-responsive section.

The predetermined condition is, for example, a condition that the sample exceeds a predetermined threshold value. Specifically, the threshold value is a statistic of the electromagnetic time series within a predetermined section. The statistic is, for example, a sum of a predetermined representative value and a predetermined dispersion degree. The statistic may be, for example, a difference between a predetermined representative value and a predetermined dispersion degree. The representative value is, for example, an average value. The dispersion degree is, for example, a standard deviation.

However, the electromagnetic time series sample may instantaneously exceed the threshold value. Therefore, in the non-responsive section sample determination processing, it may be determined whether or not a predetermined condition for the number of times the sample continuously crosses the threshold value is satisfied. In the non-responsive section sample determination processing, in a case where a predetermined condition for the number of times the sample continuously crosses the threshold value is satisfied, it is determined that the sample belongs to the non-responsive section.

The threshold value may be, for example, an electromagnetic statistic acquired by the statistic calculation processing.

For simplicity of explanation, the abnormal state estimation system 100 will be described by exemplifying a case where the non-responsive section sample determination processing is processing of determining which sample belongs to the non-responsive section among the electromagnetic time series samples based on the electromagnetic statistic acquired by the statistic calculation processing. In addition, in a case where the non-responsive section sample determination processing is processing of determining that a sample satisfying a predetermined condition is a sample belonging to the non-responsive section, the statistic calculation processing does not necessarily need to be executed.

The out-of-range data determination processing is executed on the sample (hereinafter referred to as “non-responsive section sample”) determined to be the sample belonging to the non-responsive section by the non-responsive section sample determination processing.

The out-of-range data determination processing is processing of determining whether or not each value of the non-responsive section sample is out of the range (hereinafter referred to as a “threshold region”) corresponding to each time position. The time position is a position in the time axis direction of each sample of the electromagnetic time series. Hereinafter, the non-responsive section sample of which the value (that is, the cardiac state quantity) is determined to be out of the range of the threshold region by the out-of-range data determination processing is referred to as out-of-range data.

The threshold region is a range having at least an upper limit value and a lower limit value. The upper limit value of the threshold region is hereinafter referred to as an upper threshold value. The lower limit value of the threshold region is hereinafter referred to as a lower threshold value.

The threshold region is determined for each unit processing period according to the distribution of the non-responsive section samples within the unit processing period. The upper threshold value is, for example, (M+V) in a case where the average value of the electromagnetic quantity indicated by the sample of the non-responsive section within the unit processing period including the time position at which the threshold region is determined is M and the standard deviation is V. The lower threshold value is, for example, (M−V) in a case where the average value of the electromagnetic quantity indicated by the sample of the non-responsive section within the unit processing period is M and the standard deviation is V.

Note that the upper threshold value and the lower threshold value are not necessarily limited to the sum or difference of the average value M and the standard deviation V. The upper threshold value and the lower threshold value may be a sum or a difference of values obtained by multiplying the standard deviation V by a constant (correction value) and performing adjustment according to the detection sensitivity of the electrode 101. The upper threshold value and the lower threshold value may be a result of conversion by a predetermined function using the average value M and the standard deviation V as independent variables.

In addition, the upper threshold value and the lower threshold value may be calculated based on the variance or gradient of the cardiac state quantity. The upper threshold value and the lower threshold value may be calculated based on an amount of adjustment based on a device/environmental data other than the biosignal, or continuity (presence or absence of missing of the observation value). Being out of the range of the threshold region means that the value is either less than the lower threshold value or greater than the upper threshold value.

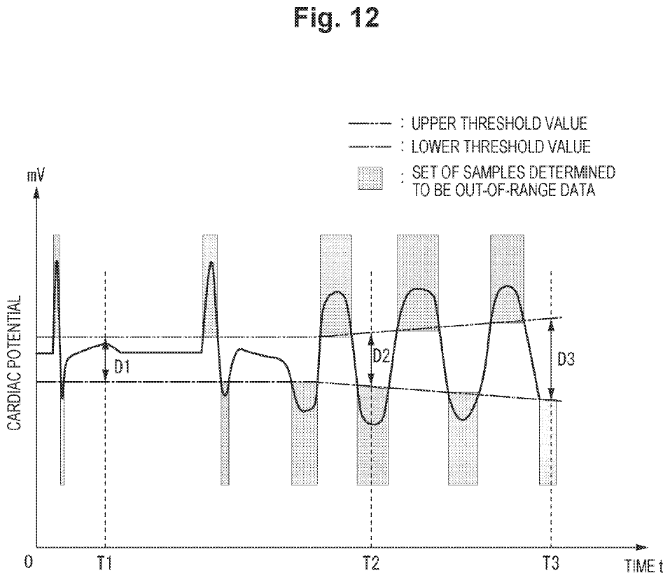

FIG. 12 is a diagram illustrating an upper threshold value, a lower threshold value, a threshold region, and out-of-range data according to the embodiment. FIG. 12 illustrates cardiac potential time series as an example of an electromagnetic time series. In FIG. 12, the horizontal axis represents the elapsed time from the time of the origin. In FIG. 12, the vertical axis represents the cardiac potential. FIG. 12 illustrates an upper threshold value and a lower threshold value. The upper threshold value and the lower threshold value in the example of FIG. 12 are examples of values calculated using cardiac potential data last 2 seconds. Therefore, as illustrated in FIG. 12, the upper threshold value and the lower threshold value are not necessarily the same at all times.

In FIG. 12, the ranges of the cardiac potential indicated by D1, D2, and D3 are threshold regions at time T1, time T2, and time T3, respectively. As illustrated in FIG. 12, the range of the cardiac potential indicated by the threshold region is not necessarily the same at all times. FIG. 12 illustrates a set of samples of the non-responsive section, which are determined as out-of-range data.

The ventricular abnormality determination processing is processing of estimating the state of the ventricle based on the sample determined as the out-of-range data by the out-of-range data determination processing. The ventricular abnormality determination processing is processing of determining that the ventricular state is abnormal when a condition indicating a predetermined appearance manner of the peak period and a condition indicating an appearance manner of the peak period in a case where the ventricular state is an abnormal state (hereinafter referred to as “peak period appearance condition”) is satisfied.

The peak period is a peak determination target period in which the out-of-range data integration time exceeds the threshold time. The out-of-range data integration time is a value obtained for each peak determination target period. The out-of-range data integration time is a value of integration of occurrence times of samples determined to be out-of-range data by the out-of-range data determination processing among the samples in each peak determination target period. That is, the out-of-range data integration time is a result obtained by assigning a predetermined time width to each sample and multiplying the time width by the number of samples determined to be out-of-range data among the samples within each peak determination target period.

The peak determination target period is a period having a predetermined length. The start time of the peak determination target period is a time that satisfies a predetermined condition. The start time of the peak determination target period is, for example, the end time of the immediately preceding peak determination target period. The start time of the peak determination target period may be, for example, a condition that the start time is a time when a predetermined time has elapsed from the immediately preceding peak determination target period.

The condition that it is the time when the predetermined time has elapsed from the immediately preceding peak determination target period means that the peak determination target period is periodically set in the ventricular abnormality determination processing. In the ventricular abnormality determination processing, for example, first, it is determined whether or not 0 milliseconds to 200 milliseconds of the electromagnetic time series are set as the peak determination target period and are the peak period. In the ventricular abnormality determination processing, next, processing of setting a period having a length of 200 milliseconds, which is subsequent to the time of 200 milliseconds being set as new 0 milliseconds, as a new peak determination target period is repeated.

The threshold time is a predetermined reference time and is a reference time for detecting a value that does not occur in the cardiac state time series of the normal heart. More specifically, the threshold time is a predetermined reference time, which is longer than the out-of-range data integration time in the cardiac state time series of the normal heart. Since the threshold time is longer than the out-of-range data integration time in the cardiac state time series of the normal heart, the peak determination target period in which the out-of-range data integration time exceeds the threshold time is a period in which the cardiac state time series of the abnormal heart appears.

The length of the peak period is 15 milliseconds, which is three times 5 milliseconds, for example, in a case where the cardiac state time series is acquired at a sampling rate of 200 Hz and three points are out-of-range data. Note that the time interval of each sample in the time series at the sampling rate of 200 Hz is 5 milliseconds. In a case where the cardiac state time series was acquired at a sampling rate of 200 Hz, the time of occurrence of the sample, that is, the predetermined time width imparted to the sample, would be, for example, 5 milliseconds.

The length of the peak determination target period is desirably substantially the same as the length of one beat of the heartbeat. Therefore, the length of the peak determination target period is, for example, 200 milliseconds.

The threshold time is, for example, a time longer than the generation time of the R wave of the normal heart. The time longer than the generation time of the R wave of the normal heart is, for example, 50 milliseconds.

An example of processing executed in the ventricular abnormality determination processing in a case where the threshold time is 50 milliseconds, the length of the peak determination target period is 200 milliseconds, and the peak determination target period is periodically set every 200 milliseconds will be specifically described. In this case, in the ventricular abnormality determination processing, processing of determining, as a peak period, a peak determination target period in which the cumulative time of the non-responsive section sample in which the electromagnetic quantity is determined to be out of the threshold region is 50 milliseconds or more among the peak determination target periods that are periodically repeated at intervals of 200 milliseconds is executed.

The peak period appearance condition is, for example, a condition that a predetermined number of peak periods continuously appear. When the peak period appearance condition is a condition that a predetermined number of peak periods continuously appear, the heart of the estimation target 9 is in a state where ventricular flutter or ventricular fibrillation has occurred. The number of continuous peak periods is a predetermined value set in advance, but is desirably a value determined in consideration of, for example, a balance between the frequency of erroneous determination and the time required to obtain a determination result.

More specifically, the value is desirably a value that achieves both the low frequency of erroneous determination and the short time required to obtain the determination result. The time required to obtain the determination result is desirably, for example, a time in which damage that may occur when the heart of the estimation target 9 is in an abnormal state can be prevented by notification. The number of continuous peak periods is, for example, five.

As described above, the first-type cardiac state estimation processing is processing of estimating the state of the heart of the estimation target 9 based on the generation time of the out-of-range data.

### (Application Scene)

An example of an application scene of the abnormal state estimation system 100 will be described. The abnormal state estimation system 100 can prevent a serious accident by being used to detect a cardiac function abnormality of a person who may develop into a serious accident when losing consciousness during work, such as a driver of a vehicle having high publicness such as a bus or a train.

By applying the abnormal state estimation system 100 to a person who uses the electric movement assist device due to disability, rehabilitation, illness, injury, or the like, it is possible to prevent an accident that occurs when the person loses consciousness during use of the electric movement assist device.

The abnormal state estimation system 100 may be used to detect a sign of sudden death during exercise. For example, as illustrated in FIG. 1, the abnormal state estimation system 100 is applied to a driver of the automobile 90.

There is a case where an in-vehicle sensor already used in the market is mounted on the automobile 90. The in-vehicle sensor is an example of the environment sensor 3. In such a case, when an abnormality occurs in the heart of the driver of the automobile 90, the abnormal state estimation system 100 can comprehensively determine the situation by taking into account the estimation reliability and data including other in-vehicle sensors.

In such a case, the abnormal state estimation system 100 gives a notification to, for example, an operator or a control system of a notification destination. The operator or control system of the notification destination can perform an action such as a call or an alarm on the driver of the automobile 90 as necessary. The control system of the operator or the notification destination may directly notify the automobile 90 of a control signal for deceleration or stop processing.

### Modification Example

In the electrode event occurrence determination processing, it may be determined whether or not an electrode event has occurred based on the environment information. For example, the electrode event occurrence determination processing may include body motion detection processing, and in the electrode event occurrence determination processing, it may be determined whether or not an electrode event has occurred based on an execution result of the body motion detection processing. For simplicity of explanation, the body motion detection processing will be described with an example in which the environmental quantity is a quantity indicating the state of the body motion of the estimation target 9.

The body motion detection processing performs body motion determination based on the environment information. The body motion determination is processing of determining a state where the body motion detection condition is satisfied as a body motion event. The body motion detection condition is a condition that the environmental quantity exceeds a predetermined threshold value. The predetermined threshold value used for the body motion detection condition is, for example, a statistic indicating a change in the environmental quantity within a predetermined time. The predetermined threshold value used for the body motion detection condition is, for example, an average value of the environmental quantity within a predetermined time. The predetermined threshold value used for the body motion detection condition may be a predetermined value.

The predetermined threshold value used for the body motion detection condition may be an output of a predetermined function that is a function having environment information included in a predetermined period as an input and has an environment output amount as an output. The environmental output amount is an amount indicating a time series state of the environmental quantity in a predetermined period. The body motion detection processing includes environment signal abnormality amplitude determination processing, environment signal abnormality standard deviation determination processing, and body motion state determination processing.

The environment signal abnormality amplitude determination processing is processing of determining the timing at which the environmental quantity indicated by the environment information exceeds a predetermined upper limit value and the timing at which the environmental quantity falls below a predetermined lower limit value as the timing of the candidate (hereinafter referred to as “body motion event candidate”) for the body motion event.

The upper limit value and the lower limit value in the environment signal abnormality amplitude determination processing may be predetermined values or values determined based on the environment information.

The upper limit value and the lower limit value in the environment signal abnormality amplitude determination processing are, for example, absolute values of the environmental quantity. The upper limit value and the lower limit value in the environment signal abnormal amplitude determination processing may be, for example, relative values from a reference value calculated from the average behavior of the time series of the environmental quantity in a predetermined period immediately before the environment signal abnormality amplitude determination processing is executed. The predetermined period is, for example, a period of several seconds. The predetermined period is a period of several tens of seconds.

The reference value may be, for example, an average value of the reference values in a predetermined period. However, the reference value is not limited to the average value as long as the reference value is an amount based on the reference value for the predetermined period.

The environment signal abnormality standard deviation determination processing is processing of determining the timing at which the deviation of the environmental quantity exceeds a predetermined threshold value as the timing of the body motion event candidate.

The predetermined threshold value in the environment signal abnormality standard deviation determination processing may be a predetermined value or a value determined based on the environment information.

The predetermined threshold value in the environment signal abnormality standard deviation determination processing is, for example, an absolute value of the deviation of the environmental quantity. The predetermined threshold value in the environment signal abnormality standard deviation determination processing may be, for example, a relative value from a reference value calculated from the average behavior of deviations of environmental quantities in a predetermined period immediately before the environment signal abnormality amplitude determination processing is executed. The predetermined period is, for example, a period of several seconds. The predetermined period is a period of several tens of seconds.

The reference value may be, for example, an average value of the reference values in a predetermined period. However, the reference value is not limited to the average value as long as the reference value is an amount based on the reference value for the predetermined period.

The body motion state determination processing is processing of determining that the timing at which both the environment signal abnormality amplitude determination processing and the environment signal abnormality standard deviation determination processing are determined to be the timing of the body motion event candidate is the timing of occurrence of the body motion event.

In the electrode event occurrence determination processing, in a case where it is determined that body motion is detected in the body motion detection processing, it may be determined that an electrode event has occurred.

The description of the body motion detection processing ends here. In the electrode event occurrence determination processing, both the electrode acquisition information and the environment information may be used in determining the occurrence of the electrode event. In such a case, the electrode event occurrence determination processing further executes body motion detection processing in addition to the biosignal abnormality amplitude determination processing, the biosignal abnormality standard deviation determination processing, and the electrode state determination processing.

In a case where both the electrode acquisition information and the environment information are used in determining the occurrence of the electrode event, in the determination of the occurrence of the electrode event, when it is determined that the sample exceeds the threshold value in at least one of the biosignal abnormality amplitude determination processing, the biosignal abnormality standard deviation determination processing, and the body motion detection processing, it is determined that the electrode event has occurred. Note that the threshold values in the biosignal abnormality amplitude determination processing are the predetermined upper limit value and the predetermined lower limit value in the biosignal abnormality amplitude determination processing, which are described above.

The initial value of the estimation reliability may be a predetermined value, or may be a value obtained by execution of predetermined processing based on the latest electrode acquisition information. The predetermined processing is, for example, calibration processing. The calibration processing is processing of changing the initial value of the estimation reliability according to the number of times the determination results is true in at least one of the abnormality amplitude determination processing, the biosignal abnormality standard deviation determination processing, and the body motion detection processing on the electromagnetic time series biosignal acquired in the calibration period. In the calibration processing, when the number of times the determination result is true in a plurality of pieces of processing among the abnormality amplitude determination processing, the biosignal abnormality standard deviation determination processing, or the body motion detection processing at the same time ta, it is determined that the number of times the determination result is true at time ta is two. The calibration period is a predetermined period after the electrode 101 is attached to the estimation target 9.

In the electrode event occurrence determination processing and the cardiac abnormality event occurrence determination processing, another statistic indicating the distribution of samples of time series may be used instead of the deviation of the distribution of samples of time series. The statistic may be, for example, a variance value, an average value, a median value, an absolute deviation, a root mean square value, a percentile value, a maximum value, or a minimum value.

The upper limit value or the lower limit value used in the electrode event occurrence determination processing and the cardiac abnormality event occurrence determination processing may dynamically change in real time based on an input from the outside or a history of past data.

The estimation reliability may be lowered by different lowering widths based on the contents of the detected abnormality. The pace of increase in the estimation reliability may change according to the content of the detected abnormality or the content indicated by the environment information. The pace is a change amount per unit time.

With respect to the setting of the peak determination target period, a new peak determination target period from 0 milliseconds may be set every time the data is updated in accordance with the sampling rate, and each peak determination target period may be set to overlap.

The peak period appearance condition does not necessarily include a condition that the peak period is continuous. Therefore, the peak period appearance condition may be a condition that the peak period occurs 4 times or more regardless of whether the peak period is continuous or discontinuous within 1000 milliseconds, for example.

### <Second-Type Cardiac State Estimation Processing>

The cardiac state estimation processing executed by the monitoring device 4 may be a second-type cardiac state estimation processing instead of the first-type cardiac state estimation processing. That is, the monitoring device 4 may estimate the state of the heart of the estimation target 9 by executing the second-type cardiac state estimation processing on the electromagnetic time series indicated by the electrode acquisition information.

The second-type cardiac state estimation processing is processing of estimating the state of the heart of the estimation target 9 based on the time interval RRI (RR-Interval) of the R waves in the electromagnetic time series.

The second-type cardiac state estimation processing is processing of estimating that the state of the heart of the estimation target 9 is abnormal, for example, when RRI in the electromagnetic time series is smaller than an RRI lower limit threshold value which is a predetermined threshold value. The RRI lower limit threshold value is, for example, RRI during exercise of a person whose the state of the heart is normal. The value larger than the RRI during exercise of a person whose the state of the heart is normal is, for example, 600 ms.

When the RRI lower limit threshold value is the RRI during exercise of a person whose the state of the heart is normal, and the RRI of the electromagnetic time series is smaller than the RRI lower limit threshold value, the possibility of occurrence of ventricular tachycardia is high. Therefore, by estimating the state of the heart depending on whether RRI in the electromagnetic time series is smaller than the RRI lower limit threshold value, it is possible to estimate whether or not the heart of the estimation target 9 is in an abnormal state where ventricular tachycardia occurs.

In the second-type cardiac state estimation processing, when RRI in the electromagnetic time series is larger than an RRI upper limit threshold value that is a predetermined threshold value different from the RRI lower limit threshold value, the state of the heart of the estimation target 9 may be estimated to be abnormal. When the state of the heart is abnormal, the activity of the heart may decrease and the pulse rate may decrease. That is, when the state of the heart is abnormal, bradycardia may occur.

By estimating the state of the heart depending on whether RRI in the electromagnetic time series is larger than the RRI upper limit threshold value, it is possible to estimate whether or not the heart of the estimation target 9 is in an abnormal state where bradycardia occurs. The RRI upper limit threshold value is preferably a value with which the occurrence of bradycardia can be estimated, and is desirably, for example, 1000 ms or more.

In the second-type cardiac state estimation processing, the state of the heart of the estimation target 9 may be estimated using the RRI lower limit threshold value and the RRI upper limit threshold value.

The second-type cardiac state estimation processing may be processing of estimating the state of the heart of the estimation target 9 by further using environment information. The environment information used by the second-type cardiac state estimation processing for estimating the state of the heart of the estimation target 9 is, for example, information acquired by an inertial sensor such as an acceleration sensor or a gyro sensor, and is information indicating acceleration of the estimation target 9 (hereinafter referred to as “detection target acceleration information”). That is, in a case where the second-type cardiac state estimation processing uses the environment information for the state of the heart of the estimation target 9, the environment sensor 3 as a source of the environment information is, for example, an inertial sensor.

Even when the state of the heart of the estimation target 9 is not in the state where the ventricular tachycardia occurs and is normal, the RRI decreases when the estimation target 9 performs exercise. Therefore, the second-type cardiac state estimation processing using not only the electromagnetic time series but also the detection target acceleration information can estimate the state of the heart of the estimation target 9 with higher accuracy than the second-type cardiac state estimation processing based only on the electromagnetic time series.

In the second-type cardiac state estimation processing using not only the electromagnetic time series but also the detection target acceleration information, normality determination is performed. The normality determination is processing of determining that the state of the heart of the estimation target 9 is normal in a case where the statistic obtained from the detection target acceleration information exceeds a threshold value satisfying a predetermined condition even in a case where the RRI obtained from the electromagnetic time series becomes smaller than a predetermined reference.

Hereinafter, the predetermined condition satisfied by the threshold value in the normal determination is referred to as a normal threshold condition. The normal threshold condition is, for example, a condition of a predetermined value. In such a case, the threshold value satisfying the normal threshold condition is a predetermined value. The normality determination determines that the state of the heart of the estimation target 9 is abnormal in a case where the RRI obtained from the electromagnetic time series becomes smaller than a predetermined reference, and only in a case where the statistic calculated based on the detection target acceleration information does not exceed a threshold value satisfying the normal threshold condition.

This is because, even when the RRI decreases, in a case where the statistic obtained from the detection target acceleration information is large, there is a high possibility that the decrease in RRI was caused by movement or exercise of the estimation target 9 rather than by the state abnormality of the heart.

The statistic obtained from the detection target acceleration information is specifically information acquired by the inertial sensor, and is a statistic of distribution of values of each sample indicated by a time series of information indicating the acceleration of the estimation target 9.

The statistic in the normality determination is, for example, a value obtained by integrating absolute values of acceleration values of three axes in a predetermined constant time. However, the statistic in the normality determination may be any value as long as the statistic is a statistic calculated based on the detection target acceleration information, and is not limited to a value obtained by integrating absolute values of acceleration values of the three axes in a predetermined constant time.

The normal threshold condition is not necessarily a condition of a predetermined value. The normal threshold condition may be, for example, a statistic calculated based on the detection target acceleration information in the past time section satisfying a predetermined condition regarding the period (hereinafter referred to as “normality determination period condition”). The normality determination period condition is, for example, a condition of three seconds before.

The normal threshold condition may be, for example, a condition that the normal threshold condition is a value of an objective variable of a predetermined function having the detection target acceleration information in the past time section satisfying the normal determination period condition as an explanatory variable.

The environment information used by the second-type cardiac state estimation processing for estimating the state of the heart of the estimation target 9 may include, for example, position information of the estimation target 9. The position information of the estimation target 9 is, for example, information acquired using a technology for acquiring position information such as a global positioning system (GPS). That is, the environment sensor 3 that acquires the position information is a device that acquires the position information of the estimation target 9 using a technology of acquiring position information such as GPS of a smartphone or the like equipped with a GPS function, for example.

When the estimation target 9 is driving the automobile 90, strenuous exercise is not often performed. Therefore, the decrease in RRI when the information indicating that the estimation target 9 is on the roadway or the information indicating that the estimation target 9 is moving at a speed equal to or higher than the walking speed such as 50 km/h is acquired based on the position information is not the decrease in RRI caused by the exercise of the estimation target 9.

Therefore, the decrease in RRI when the information indicating that the estimation target 9 is on the roadway or the information indicating that the estimation target 9 is moving at a speed equal to or higher than the walking speed such as 50 km/h is acquired based on the position information means that the probability that the state of the heart of the estimation target 9 is abnormal is high. Therefore, in a case where the second-type cardiac state estimation processing estimates the state of the heart of the estimation target 9 also based on the position information, the state of the heart of the estimation target 9 can be estimated with higher accuracy than in a case where the state of the heart of the estimation target 9 is estimated without using the position information.

### <Third-Type Cardiac State Estimation Processing>

The cardiac state estimation processing executed by the monitoring device 4 may be a third-type cardiac state estimation processing instead of the first-type cardiac state estimation processing. That is, the monitoring device 4 may estimate the state of the heart of the estimation target 9 by executing the third-type cardiac state estimation processing on the electromagnetic time series indicated by the electrode acquisition information.

The third-type cardiac state estimation processing is processing of executing the first-type cardiac state estimation processing, the second-type cardiac state estimation processing, and first integrated estimation processing. The first integrated estimation processing is processing of estimating the state of the heart of the estimation target 9 based on the estimation result of the first-type cardiac state estimation processing and the estimation result of the second-type cardiac state estimation processing.

In ventricular fibrillation which is one of phenomena caused by abnormality of the heart, the QRA waveform is irregular (refer to Reference Literature 1).

Reference Literature 1: “Jiji Medical, ventricular tachycardia and ventricular fibrillation”, [online], [retrieved on Feb. 10, 2021], Internet <https://medical.jiji.com/medical/012-1027>

In the third-type cardiac state estimation processing, the first integrated estimation processing is executed after the first-type cardiac state estimation processing and the second-type cardiac state estimation processing are executed. In the first integrated estimation processing, the state of the heart of the estimation target 9 is estimated to be abnormal only in a case where both the first-type cardiac state estimation processing and the second-type cardiac state estimation processing estimate that the state of the heart of the estimation target 9 is abnormal.

Therefore, in the first integrated estimation processing, for example, the state of the heart of the estimation target 9 is estimated to be normal in a case where the estimation result by the execution of the second-type cardiac state estimation processing estimates that the state of the heart is normal even in a case where the state of the heart is estimated to be abnormal by the execution of the first-type cardiac state estimation processing.

As described above, the third-type cardiac state estimation processing is processing of estimating the state of the heart of the estimation target 9 using not only the estimation result of either the first-type cardiac state estimation processing or the second-type cardiac state estimation processing but also the estimation results of both the first-type cardiac state estimation processing and the second-type cardiac state estimation processing.

Therefore, the third-type cardiac state estimation processing can estimate the state of the heart of the estimation target 9 with higher accuracy than a case where the state of the heart of the estimation target 9 is estimated using the estimation result of either the first-type cardiac state estimation processing or the second-type cardiac state estimation processing. Since the third-type cardiac state estimation processing estimates the state of the heart of the estimation target 9 based on two conditions of the generation of the signal and the irregularity of the QRS waveform in the non-responsive section, the state of the heart of the estimation target 9 can be estimated with high accuracy.

Note that generation of a signal in the non-responsive section means that a peak period appearance condition is satisfied.

Note that, in the second-type cardiac state estimation processing executed in the third-type cardiac state estimation processing, it is not always necessary to estimate the state of the heart of the estimation target 9 depending on whether or not the RRI exceeds the threshold value, and the state of the heart of the estimation target 9 may be estimated using the statistic of the RRI. That is, in the third-type cardiac state estimation processing, the state of the heart of the estimation target 9 may be estimated by estimating the irregularity of the QRS waveform in the ventricular fibrillation using the statistic of the RRI as the irregularity of the RRI.

The statistic of RRI may be, for example, an average of RRI, a deviation, a variance value, a median value, an absolute deviation, a root mean square value, a percentile value, a maximum value, or a minimum value.

For example, in a case where the statistic of the RRI is the average of the RRI, in the third-type cardiac state estimation processing, in a case where the difference between the repeatedly calculated average values of the RRO exceeds a predetermined threshold value, when the occurrence of a signal in the non-responsive section is confirmed, the state of the heart of the estimation target 9 is estimated to be abnormal.

The statistic of the RRI may be a statistic other than the average such as a deviation. Even in a case where the statistic of the RRI is another statistic, the state of the heart of the estimation target 9 is estimated to be abnormal depending on whether or not the difference in the repetitively calculated statistic exceeds a predetermined threshold value.

### <Fourth-Type Cardiac State Estimation Processing>

The cardiac state estimation processing executed by the monitoring device 4 may be a fourth-type cardiac state estimation processing instead of the first-type cardiac state estimation processing. That is, the monitoring device 4 may estimate the state of the heart of the estimation target 9 by executing the fourth-type cardiac state estimation processing on the electromagnetic time series indicated by the electrode acquisition information.

The fourth-type cardiac state estimation processing is processing of executing the first-type cardiac state estimation processing, the second-type cardiac state estimation processing, and second integrated estimation processing. The second integrated estimation processing is processing of estimating the state of the heart of the estimation target 9 based on the estimation result of the first-type cardiac state estimation processing and the estimation result of the second-type cardiac state estimation processing.

The second integrated estimation processing is processing of estimating that the state of the heart of the estimation target 9 is abnormal regardless of the estimation result of the first-type cardiac state estimation processing in a case where the estimation result of the second-type cardiac state estimation processing is an abnormal result. In this respect, the first integrated estimation processing and the second integrated estimation processing are different.

The second integrated estimation processing is processing of estimating that the state of the heart of the estimation target 9 is abnormal in a case where the estimation result of the second-type cardiac state estimation processing is normal even in a case where the estimation result of the first-type cardiac state estimation processing is abnormal. The second integrated estimation processing is processing of estimating that the state of the heart of the estimation target 9 is normal in a case where the estimation result of the first-type cardiac state estimation processing is normal, and in a case where the estimation result of the second-type cardiac state estimation processing is normal.

In the fourth-type cardiac state estimation processing, the second integrated estimation processing is executed after the first-type cardiac state estimation processing and the second-type cardiac state estimation processing are executed.

As described above, the fourth-type cardiac state estimation processing is processing of estimating the state of the heart of the estimation target 9 using not only the estimation result of either the first-type cardiac state estimation processing or the second-type cardiac state estimation processing but also the estimation results of both the first-type cardiac state estimation processing and the second-type cardiac state estimation processing.

Therefore, the fourth-type cardiac state estimation processing can estimate the state of the heart of the estimation target 9 with higher accuracy than a case where the state of the heart of the estimation target 9 is estimated using the estimation result of either the first-type cardiac state estimation processing or the second-type cardiac state estimation processing.

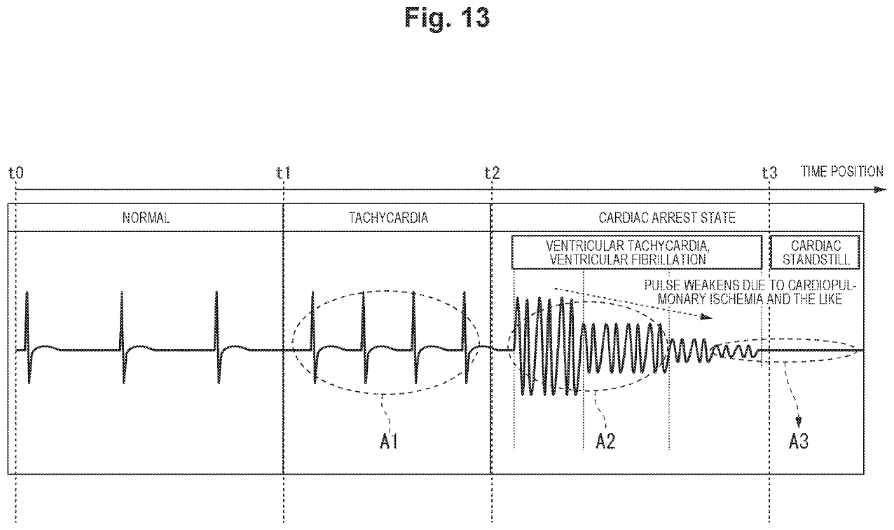

FIG. 13 is an explanatory diagram for describing an effect obtained by the fourth-type cardiac state estimation processing according to the modification example. The vertical axis in FIG. 13 represents the potential. FIG. 13 illustrates a process in which ventricular fibrillation occurs after ventricular tachycardia occurs in the estimation target 9 in which a normal cardiac potential has occurred. FIG. 13 illustrates that the heart is normal in the period from time position t0 to time position t1. FIG. 13 illustrates that tachycardia occurs in a period from time position t1 to time position t2. FIG. 13 illustrates that the ventricular flutter or the ventricular fibrillation occurs in the period from time position t2 to time position t3. The waveform in the period from time t2 to time t3 in FIG. 13 is an example of a waveform indicating that the pulse becomes weak due to, for example, cardiopulmonary ischemia or the like. FIG. 13 illustrates transition to the state of cardiac arrest after time position t3.

A waveform surrounded by a frame A1 in FIG. 13 is an example of a waveform estimated to be abnormal by the second-type cardiac state estimation processing. A waveform surrounded by a frame A2 in FIG. 13 is an example of a waveform estimated to be abnormal by the first-type cardiac state estimation processing. A waveform surrounded by region A3 in FIG. 13 is an example of a waveform leading to cardiac arrest.

### <Fifth-Type Cardiac State Estimation Processing>

The cardiac state estimation processing executed by the monitoring device 4 may be a fifth-type cardiac state estimation processing instead of the first-type cardiac state estimation processing. That is, the monitoring device 4 may estimate the state of the heart of the estimation target 9 by executing the fifth-type cardiac state estimation processing on the electromagnetic time series indicated by the electrode acquisition information.

The fifth-type cardiac state estimation processing is different from the first-type cardiac state estimation processing to the fourth-type cardiac state estimation processing in that a state of cardiac arrest is estimated. The state of the cardiac arrest is, for example, a state after time t3 in FIG. 13. The fifth-type cardiac state estimation processing is processing of executing the first-type cardiac state estimation processing, the second-type cardiac state estimation processing, the first integrated estimation processing, the second integrated estimation processing, and cardiac arrest estimation processing. In the fifth-type cardiac state estimation processing, the first integrated estimation processing and the second integrated estimation processing are executed after the execution of the first-type cardiac state estimation processing and the second-type cardiac state estimation processing, and then the cardiac arrest estimation processing is executed.

The cardiac arrest estimation processing is processing of estimating that the state of the heart of the estimation target 9 is the state of cardiac arrest in a case where the deviation of the distribution of the electromagnetic quantity within a predetermined period after the abnormality occurrence time position is equal to or less than a predetermined threshold value. The abnormality occurrence time position is a time position at which the state of the heart of the estimation target 9 is estimated to be abnormal by the first integrated estimation processing or the second integrated estimation processing.

At the time of cardiac arrest, the variation of the cardiac potential associated with the heartbeat is substantially zero, and is substantially the same as 0 mV. Therefore, in the cardiac arrest estimation processing, for example, processing of calculating the deviation of the distribution of the electromagnetic quantity every 200 ms and processing of determining whether or not the calculated deviation is out of the range of ±15 mV are executed.

Therefore, the cardiac arrest can be detected by executing the fifth-type cardiac state estimation processing.

In the cardiac state estimation processing, the waveform of the electromagnetic time series may be shaped using a filter that performs various types of signal processing such as an analog filter, a digital filter such as a finite impulse response (FIR) or an infinite impulse response (IR), and a moving average filter to which a moving average is applied. For example, a noise component included in the electromagnetic time series is removed by using the filter.

The monitoring device 4 may be implemented by using a plurality of information processing devices connected to be capable of communicating with each other via a network. In this case, the functional units included in the monitoring device 4 may be implemented in a distributed manner in the plurality of information processing devices.

The control device 5 may be implemented by using a plurality of information processing devices connected to be capable of communicating with each other via a network. In this case, the functional units included in the control device 5 may be implemented in a distributed manner in the plurality of information processing devices.

Note that the monitoring device 4 and the control device 5 are not necessarily mounted as different devices. The monitoring device 4 and the control device 5 may be implemented as one device having both functions, for example. For example, the control unit 41 may include the notification determination unit 520.

Note that, all or some of the functions of the abnormal state estimation system 100 may be realized using hardware such as an application specific integrated circuit (ASIC), a programmable logic device (PLD), or a field programmable gate array (FPGA). The program may be recorded on a computer-readable recording medium. The computer-readable recording medium is a storage device such as a portable medium (for example, a flexible disk, a magneto-optical disc, a ROM, or a CD-ROM), or a hard disk built in a computer system. The program may be transmitted via an electrical communication line.

Note that the monitoring device 4 is an example of a state estimation device.

Although the embodiments of the present invention have been described in detail with reference to the drawings, specific configurations are not limited to the embodiments, and include design and the like within the scope of the present invention without departing from the gist of the present invention.

### REFERENCE SIGNS LIST

100 Abnormal state estimation system
1 Biosignal acquisition device
101 Electrode
2 Relay terminal
3 Environment sensor
4 Monitoring device
5 Control device
41 Control unit
42 Input unit
43 Communication unit
44 Storage unit
45 Output unit
410 Information acquisition unit
420 Estimation unit
430 Storage control unit
440 Communication control unit
450 Output control unit
411 Electrode acquisition information acquisition unit
412 Environment information acquisition unit
421 Electrode event occurrence determination unit
422 Cardiac abnormality event occurrence determination unit
423 Estimation reliability estimation unit
51 Control unit
52 Input unit
53 Communication unit
54 Storage unit
55 Output unit
510 Result acquisition unit
520 Notification determination unit
530 Storage control unit
540 Communication control unit
550 Output control unit
91 Processor
92 Memory
93 Processor
94 Memory
9 Estimation target
90 Automobile

1. A state estimation device comprising:
an information acquisition unit, implemented using one or more computing devices, configured to acquire at least one of (i) electrode acquisition information indicating an electromagnetic time series that is a time series of an electromagnetic quantity that is an electromagnetic force, field, or energy applied to an electrode from which a signal indicating a state of a heart of an estimation target is acquirable or (ii) environment information regarding a time series of an environmental quantity that is an amount related to at least one of a state of motion of the estimation target or an environment in which the estimation target is present; and
an estimation unit, implemented using one or more computing devices, configured to use at least one of the electrode acquisition information or the environment information to determine whether or not an electrode event, which is an abnormality in acquisition of a signal indicating the state of the heart of the estimation target by the electrode, has occurred.

2. The state estimation device according to claim 1, wherein:
the estimation unit is configured to determine an occurrence of a cardiac abnormality event that is an abnormality of the state of the heart of the estimation target based on the electrode acquisition information, and
the estimation unit is configured to estimate an estimation reliability that is a probability of an estimation result of occurrence of the cardiac abnormality event.

3. The state estimation device according to claim 2, wherein
the estimation reliability is estimated also based on a result of determination of occurrence of the electrode event.

4. A state estimation method comprising:
acquiring at least one of (i) electrode acquisition information indicating an electromagnetic time series of an electromagnetic quantity that is an electromagnetic force, field, or energy applied to an electrode from which a signal indicating a state of a heart of an estimation target is acquirable or (ii) environment information regarding a time series of an environmental quantity that is an amount related to at least one of a state of motion of the estimation target or an environment in which the estimation target is present; and
using at least one of the electrode acquisition information or the environment information to determine whether or not an electrode event, which is an abnormality in acquisition of a signal indicating the state of the heart of the estimation target by the electrode, has occurred.

5. A non-transitory recording medium storing a program for, wherein execution of the program causes a computer to perform operations comprising:
acquiring at least one of (i) electrode acquisition information indicating an electromagnetic time series of an electromagnetic quantity that is an electromagnetic force, field, or energy applied to an electrode from which a signal indicating a state of a heart of an estimation target is acquirable or (ii) environment information regarding a time series of an environmental quantity that is an amount related to at least one of a state of motion of the estimation target or an environment in which the estimation target is present; and
using at least one of the electrode acquisition information or the environment information to determine whether or not an electrode event, which is an abnormality in acquisition of a signal indicating the state of the heart of the estimation target by the electrode, has occurred.
